(19) United States
(12) Patent Application Publication
(10) Pub. No.: US 2019/0137385 A1
(43) Pub. Date: May 9, 2019

(54) METHOD AND APPARATUS FOR MEASURING THE WATER CONCENTRATION IN A LIGHT-DIFFUSING MATERIAL

(71) Applicant: CONNECTED PHYSICS SAS, Orsay (FR)

(72) Inventors: Thomas NAPPEZ, Vanves (FR); Hadrien LEPAGE, Paris (FR)

(73) Assignee: CONNECTED PHYSICS SAS, Orsay (FR)

(21) Appl. No.: 16/096,213

(22) PCT Filed: Apr. 26, 2017
(86) PCT No.: PCT/FR2017/050992

§ 371 Date: Oct. 24, 2018

(30) Foreign Application Priority Data
Apr. 27, 2016 (FR) 1653740

(51) Int. Cl. G01N 21/31; G01N 21/47; A61B 5/00

(52) U.S. Cl. CPC G01N 21/314; G01N 21/4738; A61B 5/6833; G01N 2021/1734; A61B 5/443; A61B 5/448; A61B 5/4875; A61B 5/0077

(57) ABSTRACT

A method for measuring the water concentration in a light-diffusing material, includes the following steps: an emission by N light sources of beams with wavelengths; an acquisition by M sensors, sensitive in at least one portion of the wavelengths; wherein M+N>3. The method also includes the steps of: calculating a first piece of information representing the diffusion, and a second piece of information representing the absorption, as a function of the signals and a piece of digital information representing the diffusing material as well as the sources and the sensors; calculating the water concentration in the sample as a function of the second piece of information.

### CROSS-REFERENCE TO RELATED APPLICATIONS

This application is a National Phase Entry of International Patent Application No. PCT/FR2017/050992, filed on Apr. 26, 2017, which claims priority to French Patent Application Serial No. 16/53740, filed on Apr. 27, 2016, both of which are incorporated by reference herein.

### TECHNICAL FIELD

The present invention relates to the field of measuring the concentration of the water contained in a material, and specifically in a light-diffusing material such as human skin, plants, paper, plaster, etc.

### BACKGROUND

In general terms, the prior art discloses various families of measurements of the hydration of a material. A first family consists of indirect measurements, based on the relationship between water content and electrical conductivity, which are not applicable only when the electrical conductivity measured is considered to be an image of the water concentration, water which makes is possible to a greater or lesser extent to dilute the electrically charged particles initially found in the material to be analysed and responsible for the electrical conduction thereof.

To measure skin hydration it is possible to use corneometers, for example, the measurement of which is based on the electrical properties of the skin. A current passes between two electrodes attached to the skin. The higher the hydration of the stratum corneum, the better the electric current can pass through. Certain factors can affect the measurements: sweat, skin temperature, environmental humidity rate, state of the skin surface, etc. (a rough surface reduces the contact surface between the probe and the skin, the application of a topical that is rich in water and ionic substances can modify the measurement).

The second family of measurements is based on a direct analysis by elastic or inelastic optical spectroscopy. The present invention belongs to this second family of direct measurement. The assessment of the amount of water in organic material and biological tissue is carried out by infrared spectrophotometry. This technique uses light made up of a plurality of wavelengths to obtain the reflectance or transmission spectrum of the material to be analysed. An assessment of the water concentration in the sample is thus obtained by comparing the power received at a wavelength close to a water molecule absorption band with that received at a wavelength distant from the water molecule absorption bands.

A plurality of embodiments of this measurement technique are known:

Emission of white light onto the sample and recovery in a spectrometer, making it possible to study the spectrum of the sample in a resolved manner,

Emission of monochromatic light with different emitters used in sequence, making it possible to use a photodetector instead of a spectrometer,

these techniques can be carried out in bulk optics or in fibre optics.

U.S. Pat. No. 8,180,419 is known, for example, which describes a method for determining tissue hydration, comprising a step of detecting electromagnetic radiation diffused and reflected by the tissue. This detected electromagnetic radiation is used to determine the spectral absorption bandwidth by correlating the spectral absorption bandwidth of a tissue hydration index.

European patent application EP 2956049 is also known, which discloses a non-invasive method for determining hydration, the state of hydration, the total body water, or the water concentration by quantitative spectroscopy. This solution comprises subsystems optimised to comply with the complexities of tissue spectroscopy, with strict requirements of signal-to-noise ratio and photometric accuracy, with tissue sampling errors, maintaining calibration and with calibration transfer. The subsystems comprise a lighting subsystem, a tissue sampling subsystem, a spectrometer subsystem, a data acquisition subsystem, a computer subsystem, and a calibration subsystem.

U.S. Pat. No. 6,442,408 describes another non-invasive solution for quantifying the hydration of the stratum comeum of a living patient. Near infrared (NIR) spectral measurements are made and the NIR absorption spectra obtained are transmitted to an analyser for subsequent processing. This consists of detecting and erasing invalid spectral measurements, and of increasing the signal-to-noise ratio. A calibration model developed from an overall example of measurements is applied to predict the hydration of the sample. The method for measuring hydration provides additional information on the systematic tissue variability, namely the water content of the epidermal layer of the skin and the depth of the penetrating incident light.

These solutions based on direct measurement by spectroscopic analysis are not entirely satisfactory, since the intensity of the measured light is partially a function only of the absorption of water molecules in the analysed environment, the skin for example. Disrupting factors are involved and do not allow the spectroscopic measurement of water content to be directly deduced. None of these measurement techniques of the prior art make it possible to obtain quantitative and absolute water concentration values. Indeed, they rely on a plurality of hypotheses:

the absorption by the water of the radiation emitted at the one or more wavelengths distant from the absorption bands is negligible,

the diffusion coefficient is identical and/or the impact thereof is negligible for all the working wavelengths,

the penetration depth of the radiation into the sample is identical for all the working wavelengths and, if need be, for all the various emitters.

A plurality of prior art solutions propose to solve this problem by storing a calibration table that makes it possible to provide reprocessed information as a function of data resulting from measurements on reference samples. This solution is very complicated to implement and only makes it possible to take a measurement on materials that have been previously characterised, and in the same conditions as those used during this calibration phase.

### SUMMARY

In order to overcome such drawbacks, the present invention relates, in its most general sense, to a method for measuring the water concentration in a light-diffusing material comprising the following steps:

emission by N light sources SLᵢ of beams having Lambdaᵢ wavelengths

acquisition by M sensors Pⱼ sensitive in at least one portion of said Lambdaᵢ wavelengths

wherein M+N≥3

characterised in that it also comprises the steps of:

calculating a first piece of information I_{dif} representing the diffusion, and a second piece of information I_{abs} representing the absorption, as a function of said signals Sⱼ and a piece of digital information representing the diffusing material as well as said sources SLᵢ and said sensors Pⱼ; ideally, the piece of digital information is supplied by one or more functions which are determined i) by a calibration step by acquiring signals Sⱼ for samples having a known water content or ii) by determining diffusion and absorption coefficients from a digital model (43) for simulating the light/sample interaction;
calculating said water concentration as a function of said second piece of information I_{abs}.

The Lambdaᵢ wavelength is preferably chosen around 1450, 1940, 970 or 1190 nanometres, for the light sources and sensors outputting a signal representing the diffusion in a wavelength corresponding to a water-absorption peak. According to one alternative embodiment, the Lambdaᵢ wavelength is chosen around 1050, 1070, 1100 or 1300 nanometres, for the light sources and sensors outputting the signal representing the diffusion in a wavelength other than a water-absorption peak.

Advantageously, said piece of digital information representing the diffusing material is made up of a digital model or an analytic model obtained from optimised phantom samples or digital simulations. According to one advantageous embodiment, said piece of digital information representing said sources SLᵢ comprises information chosen among the geometry of said sources and the optical characteristics of said sources. According to one alternative embodiment, said digital information representing said sensors Pⱼ comprises information chosen among the geometry of said sources and the optical characteristics of said sensors. Ideally, the digital information is supplied by one or more functions which are determined i) by a calibration step by acquiring signals Sⱼ for samples having a known water content or ii) by determining diffusion and absorption coefficients from a digital model (43) for simulating the light/sample interaction.

The invention also relates to an apparatus for measuring the water concentration in a light-diffusing material comprising N light sources SLᵢ emitting in Lambdaᵢ wavelengths and M sensors Pⱼ sensitive in at least one portion of said Lambdaᵢ wavelengths, wherein N+M≥3, characterised in that it comprises an electronic circuit including, for each of said sensors Pⱼ, a signal-processing means for outputting a signal Sⱼ and a computer controlling an output process, as a function of said signals Sⱼ and a piece of digital information representing the diffusing material and the geometry of said sources SLᵢ and of said sensors Pᵢ, a first piece of information representing the diffusion, and a second piece of information representing the absorption, and processing to determine said water concentration in the material. The digital information is ideally supplied by one or more functions which are determined i) by a calibration step by acquiring signals Sⱼ for samples having a known water content or ii) by determining diffusion and absorption coefficients from a digital model (43) for simulating the light/sample interaction.

Advantageously, said N light sources SLᵢ and said M sensors Pⱼ are housed in a mounting opening by a window for contact with the material to be characterised, said mounting comprising a screen that prevents direct and/or reflected transmission, without prior penetration into said diffusing material, of the light from a light source towards a sensor. The window of the mounting preferably has an anti-glare treatment. According to a preferred embodiment, said N light sources SLᵢ are polarised and the direction of the emitted beam forms a Brewster angle with the optical axis of said M sensors Pⱼ.

According to a particular embodiment, the apparatus for measuring the water concentration in a diffusing material comprises radiocommunication means for periodically transmitting the result of the measurement to a remote apparatus. According to one alternative embodiment, the apparatus is made up of a patch having means for reversible connection with a patient.

The apparatus according to the invention can furthermore have the following features, independently or in combination:

it comprises at least two light source/sensor pairs, a first light source/sensor pair defining a first separation distance between the sensor and the source, and a second light source/sensor pair defining a second separation distance;

the sources of the two pairs both emit with a spectrum centred around a wavelength close to a water-absorption peak, or one of the two sources emits with a spectrum centred around a wavelength close to a water-absorption peak and the other source emits with a spectrum centred around a zero-water-absorption wavelength;

the sources of the two pairs both emit with a spectrum centred around an absorption wavelength of 1450 nm, 1500 nm or 1550 nm;

the sources of the two pairs both emit in a zero-water-absorption wavelength;

the sources of the two pairs both emit with a spectrum centred around 1300 nm;

the apparatus comprises a third light source/sensor pair, the light source emitting in a water-absorption wavelength;

the apparatus comprises a third light source/sensor pair, the light source emitting in a zero-water-absorption wavelength;

the light source emitting in a water-absorption wavelength is arranged as close as possible to the sensor;

the apparatus comprises a sensor and three sources, the three sources belonging to three separate source/sensor pairs, respectively, or comprising two sensors and two sources, one of the two sources emitting in a wavelength close to a water-absorption peak, and the other source emitting in a zero-water-absorption wavelength;

the apparatus comprises, between the two separate sources, a series of separate sensors arranged in line;

the apparatus comprises, between the two sources, a linear strip of photodiodes;

the apparatus comprises a central or off-centre sensor and, around this sensor, a series of light-emitting sources centred around absorption and/or zero-absorption wavelengths of the light;

the apparatus comprises at least one central or off-centre light-emitting source emitting with a light spectrum centred around a absorption or zero-absorption wavelength of the light and, around this source, a series of sensors;

the apparatus comprises a mask opaque to the various wavelengths used, inserted physically between a source and an adjacent sensor;

the mask forms, around each source, a tunnel for concentrating the emitted light in a specific zone.

The invention also relates to uses of the method for:

characterising the hydration of the skin,

characterising the hydration of a lock of hair,

characterising the water concentration in a cellulosic matrix [paper; cardboard; wood],

characterising the water concentration in a food matrix,

characterising the water concentration in a plant,

characterising the water concentration in a hydrocarbon,

characterising the water concentration in a chemical product or a powder,

characterising the water concentration in a medicinal product,

characterising the water concentration in a construction material,

characterising the water concentration in a building coating,

characterising the water concentration in a fabric,

characterising the water concentration in leather,

characterising the hydration of the skin and/or characterising the quality and/or the amount of collagen or keratin in the skin.

### BRIEF DESCRIPTION OF THE DRAWINGS

The present invention will be best understood upon reading the following detailed description of a non-restrictive exemplary embodiment, while referring to the appended drawings, wherein:

FIG. 1 shows a schematic view of an apparatus according to the invention;

FIG. 2 shows a schematic view of the functional architecture;

FIG. 3 shows a schematic view of the principle for calculating the water concentration;

FIG. 4 shows a cross-section of the sample, schematically showing the path of the light beams between a source and a sensor;

FIG. 5 is the result of simulating the light/sample interaction and indicates the logarithm of the signal log(S) received in the sensor as a function of the absorption coefficient á=c*μₐ of the sample and the reduced diffusion coefficient thereof μₛ′;

FIG. 6 shows a schematic view of a first alternative embodiment;

FIG. 7 shows a schematic view of a second alternative embodiment;

FIG. 8 shows a schematic view of a third alternative embodiment;

FIGS. 9 and 10 show, in perspective views, two alternative embodiments of masks for the device according to the invention;

FIG. 11 is the result of simulating the light/sample interaction and indicates the logarithm of the signal log(S) received in the sensor as a function of the absorption coefficient á=c*μₐ of the sample and the reduced diffusion coefficient thereof μₛ′ for two different sensor-emitter pairs;

FIG. 12 shows the theoretical water absorption spectrum, and the spectra obtained after the light from various sources has passed through a sample that absorbs and diffuses light, said sources having emission peaks centred around 1450 nm, 1500 nm and 1550 nm, respectively;

FIG. 13 shows a schematic view of an alternative embodiment of the principle for calculating the water concentration from digital simulations. The analytic functions fᵢ are obtained by solving the radiative transfer equation, by Monte-Carlo digital simulations, for example. These simulations model the interaction between the light and the sample, taking into account opto-geometric parameters;

FIGS. 14a to 14c show three analytic functions obtained for three separate sources, respectively, showing the signals perceived after the light from said three sources has passed through different phantom samples having known water concentration and diffusion coefficient. It is observed that these functions are in the form of curved surfaces. An unknown sample analysed by the device according to the invention provided with three sources will make it possible to detect three signals having numerical values S₁, S₂ and S₃. These three signals correspond to the representations of the corresponding functions, zones or isocontours and thus of the (water concentration, diffusion coefficient) pairs. The search for the water concentration and the diffusion coefficient of the unknown sample involves identifying whether there is a unique solution for the (water concentration, diffusion coefficient) pair that simultaneously matches the three corresponding isocontours on the three functions;

FIGS. 15a to 15c show another possible representation of these three analytic functions, on each of which is also marked by a curve, the set of (water concentration, diffusion coefficient) pairs of samples giving the same signal S₁ for the curve of FIG. 15a, S₂ for the curve of FIG. 15b and S₃ for the curve of FIG. 15c, from which the unique (water concentration, diffusion coefficient) pair found in the three curves will be deduced. As an example of the calculation of the correct (water concentration, diffusion coefficient) pair, the normalised values for these three signals are as follows: S₁=0.59; S₂=0.78; S₃=0.71;

FIG. 16 shows, with this example, a comparative table of the water concentration and diffusion coefficient results obtained according to the method of the invention (“measurement”) on a known sample, with respect to the known water concentrations and diffusion coefficient of said sample (“experimental”);

FIG. 17 shows, for this same example, the least squares minimisation method for calculating the correct (water concentration, diffusion coefficient) pair. This figure shows the variance for the three signals S₁, S₂ and S₃, which is quite minimal with the sought (water concentration=66% and diffusion coefficient=119/cm) pair;

The graphics of FIGS. 18a and 18b show the linear correlations observed between the water concentration and the diffusion coefficient measured according to the method of the invention, and the corresponding experimental values of known samples;

FIG. 19 shows two curves that depict the progression of the water concentration measured by the method according to the invention over 24 hours, of two similar skin samples from the same individual (pig), left to dry in the open air (“air” curve) and in contact with physiological liquid (“water” curve), respectively, displaying a logical progression: the measurements obtained for the sample in contact with physiological liquid are relatively constant, and those of the sample left in the open air fall with the air exposure time;

FIGS. 20a and 20b show two histograms depicting the progression over time of the measurements of water concentration according to the method of the invention, conducted on a volunteer patient (dermis of the calf), during a day with no particular sporting activity (FIG. 20a) as well as during a day interrupted by a sports session at a specific time (4:30 PM), after which the volunteer did not drink anything until 9:30 PM and then had dinner;

FIGS. 21a to 21g show different embodiments of the device for implementing the method according to the invention; and

FIG. 22 shows a schematic view of a second alternative embodiment of the principle for calculating the water concentration from phantom samples, in which the analytic functions fᵢ are obtained from phantom samples reproducing the optical behaviour of the skin.

### DETAILED DESCRIPTION

The example of an embodiment of the invention described hereunder as a non-restrictive example makes it possible to obtain a measurement of the water concentration in a light-diffusing sample in an absolute, non-relative manner, without using a spectrometer. Indeed, conventional spectrometry is a versatile technique that is not suited to the specific features of absolute measurement in a light-diffusing sample. The measurement of the light-diffusion coefficient in the organic material and the biological tissues at the working wavelength should actually be taken in order to dissociate the contribution of the diffusion from that of the absorption in the received signal.

For this purpose, the present invention suggests using spatially resolved spectroscopy (SRS) and coupling same with a step of experimental calibration or physical modelling of the interaction between the light and the sample. These models reproduce the opto-geometric parameters as well as the sample to be analysed and solve the radiative transfer equation (RTE) using Monte Carlo algorithms or the Kubelka-Munk approximation, for example. As a function of the power emitted by each source and received by each sensor, this calibration or this model makes it possible to return to the reduced diffusion coefficient and to the absorption coefficient of the sample. Since the wavelength of the emitted light is close to and around a corresponding water absorption band, this absorption coefficient reveals the water concentration in the sample.

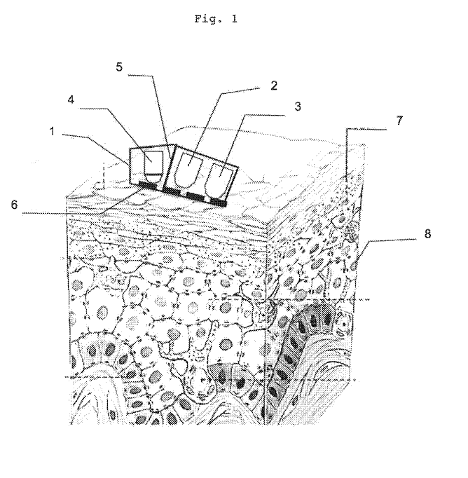

FIG. 1 shows a simplified view of an apparatus according to the invention. It is made up of a housing (1) containing two light-emitting diodes (2, 3) and a photodiode (4), aligned in the described example, and separated by a distance of several tens of micrometers to several millimetres. The light-emitting diodes (2, 3) are separated from the photodiode (4) by an opaque wall (5) preventing direct light transmission.

The housing (1) has a window closed by a transparent leaf (6) optionally coated with an anti-glare filter, the surface of which can be applied directly against the granular layer (7) of the skin (8). The housing (1) also comprises a computer and communication means and/or a display means.

The sources (2, 3) used to take the measurement are preferably light-emitting diodes (optionally covered with a lens to direct the radiation) or lasers, specifically semiconductor lasers (also referred to as laser diodes). The wavelengths used can be close to the following values: 970 nm, 1050 nm, 1190 nm, 1300 nm, 1450 nm and 1940 nm. The sensor (4) used is preferably a photodiode, which can be used in the photovoltaic or photoconductive mode. It can also be a pixel sensor (CMOS, CCD), an avalanche photodiode, a photomultiplier, a phototransistor, a photoresistor, etc.

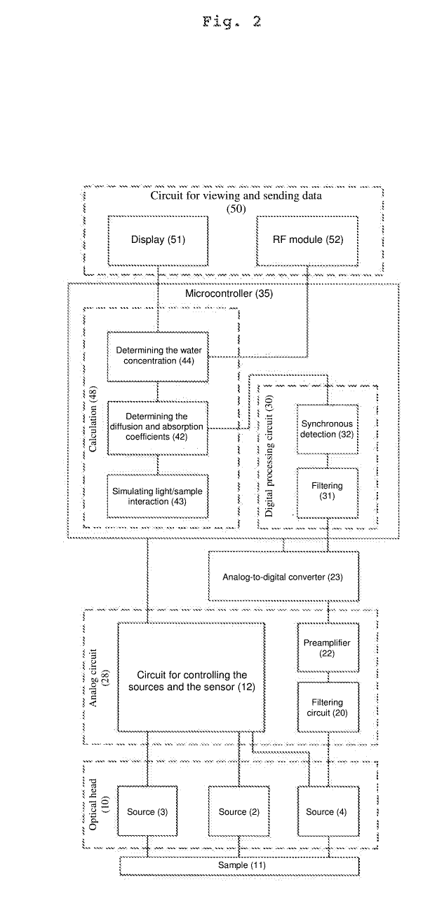

FIG. 2 depicts the functional architecture of the apparatus. It comprises an optical head (10) applied against the sample (11) to transmit the light coming from the two sources (2, 3) and to receive the light retransmitted by the sample (11) by a sensor (4). A control circuit (12) for controlling the sources (2, 3) and the sensor (4) powers the optical head (10), ensuring the sequential lighting of the sources (2, 3). In the described example, the power supply is modulated by a periodic signal (rectangular or sine-wave, for example) to facilitate the filtering and the improvement of the signal-to-noise ratio.

The signal output by the sensor (4) is processed by the analog circuit (20) comprising a filtering circuit (21) and a preamplifier (22). The signal is then digitised by an analog-to-digital converter (23). A microcontroller (35) provides functions of processing the digital signal (30) and calculations (40).

The computer controls the following in series:

a step of powering the first light-emitting diode (2) and measuring the intensity captured by the photodiode (4) and recording the signal S₁ corresponding to said intensity, followed by switching off the light-emitting diode (2);

a step of powering the second light-emitting diode (3) and measuring the intensity captured by the photodiode (4) and recording the signal S₂ corresponding to said intensity, followed by switching off the light-emitting diode (3);

a step of calculating, from the values S₁ and S₂, the piece of information linked with the diffusion I_{d} and the piece of information linked with the absorption I_{abs};

a step of calculating the water concentration from the piece of information linked with the absorption I_{abs}.

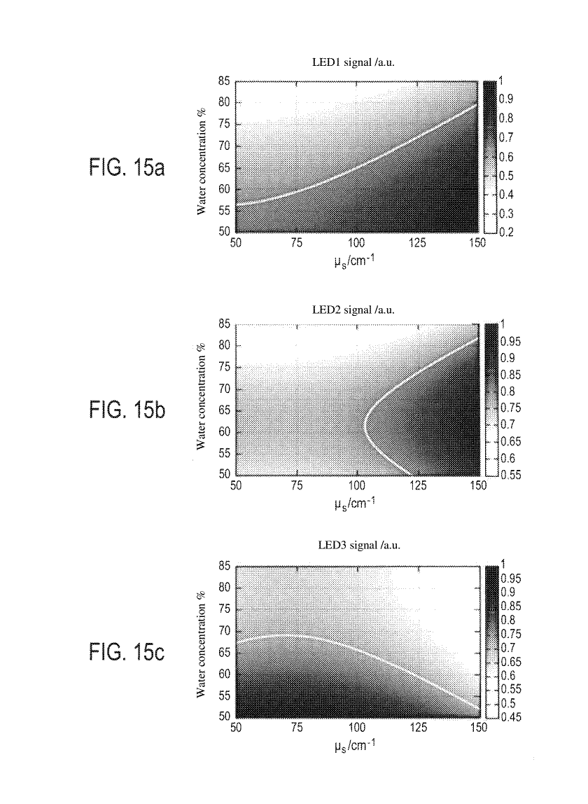

The digital signal processing (30) performs digital filtering (31) and synchronous detection (32). The computing processes (40) perform a step of determining diffusion and absorption coefficients (42) from experimental charts of a digital model (43) for simulating the light/sample interaction such as those shown in FIGS. 5, 11, 14a, 14b, 14c, 15a, 15b or 15c, and a step of determining the water concentration (44). Ideally, these coefficients are obtained from a piece of digital information provided by one or more functions which are determined i) by a calibration step by acquiring signals Sⱼ for samples having a known water content or ii) by determining diffusion and absorption coefficients from a digital model (43) for simulating the light/sample interaction. The apparatus comprises a viewing circuit (50) with a display (51) and a radiofrequency module (52).

FIG. 3 shows a block diagram of the calculations performed. The computer receives a first digital signal S₁ corresponding to the light intensity detected when lighting one of the sources (2) and a second digital signal S₂ corresponding to the light intensity detected when lighting the other source (3). These signals are processed to determine:

the reduced diffusion coefficient μₛ′

the absorption coefficient α

This processing corresponds to resolving a system of two equations with two unknowns indicated in FIG. 3, wherein the functions f are obtained using the experimental charts or the digital model wherein the function L_{eff}(μₛ′) is a prefactor and wherein the function L_{eff}(μₛ′) is the actual distance representing the path traveled by a photon in the sample before reaching the sensor. A digital or analytic model is saved in the memory. This corresponds to the result of simulating the interaction between the light and the sample, taking into account the opto-geometric parameters or the result of an experimental calibration step with samples having known absorbent concentration and diffusion coefficient parameters.

In the case of the digital model, the determination of the water concentration c is calculated by a function, is in a simplified form a linear function, for example c=α/μₐ, wherein μₐ is the water-absorption coefficient at the working wavelength. The determination of this function can be carried out by a calibration step by acquiring signals S₁ and S₂ for samples having a known water content or by calculating the actual water absorption at the spectrum emitted by the emitters.

### Other Embodiments are Contemplated

In a first alternative embodiment, shown in FIG. 6, the apparatus comprises a single source (62) having radiation close to a water absorption band and two sensors (64, 60).

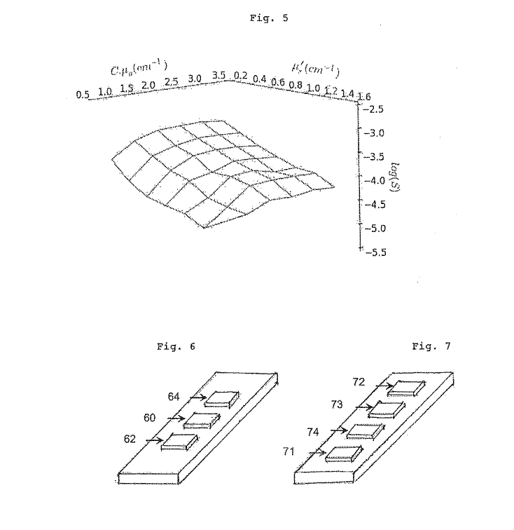

In a second alternative embodiment, shown in FIG. 7, the apparatus comprises a single source (71) having radiation close to a water absorption band, two sources (72, 73) having radiation distant from a water absorption band and one sensor (74).

The use of different distances between the sources and the sensors makes it possible to probe the sample at various depths and, using sources that have radiation close to a water absorption band, makes it possible to trace a profile of the water concentration as a function of the depth.

In a third alternative embodiment, shown in FIG. 8, the apparatus comprises a plurality of sources (or a plurality of sensors) arranged concentrically with respect to the sensor (or the source). This embodiment makes it possible to homogenise the measurements in space and to measure the reduced diffusion coefficient of the sample in a plurality of directions. In the case of human skin, the angular progression of this coefficient can be connected to characteristics linked with the ageing of the skin.

These apparatuses can have different characteristics and specific features:

Use of one or more other wavelengths to measure the water concentration using other absorption bands.

Use of one or more other wavelengths to measure the reduced diffusion coefficient at different wavelengths.

Additional moisture and temperature sensor for correlating the hydration measurement results with the ambient humidity and temperature.

Additional sensor of imperceptible water losses to measure the dynamics of water losses through the epidermis and the stratum comeum for the skin.

Additional visible colour sensor.

Positioning of the sources and the sensors according to various heights to limit the direct lighting of the one or more sensors by the closest sources.

Positioning of materials to absorb the radiation emitted by the sources to limit the direct lighting of the one or more sensors by the closest sources.

Use of the flight-time technique to measure the diffusion coefficient of the sample.

Work at the Brewster angle, lighting the sample with transverse magnetic radiation or only detecting this polarisation to limit the influence of the specular reflections on the sample.

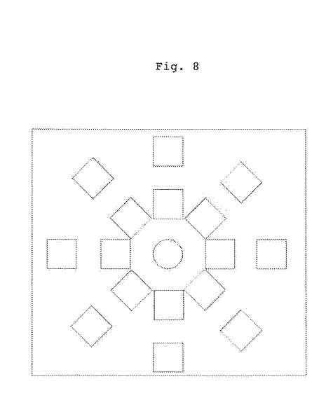

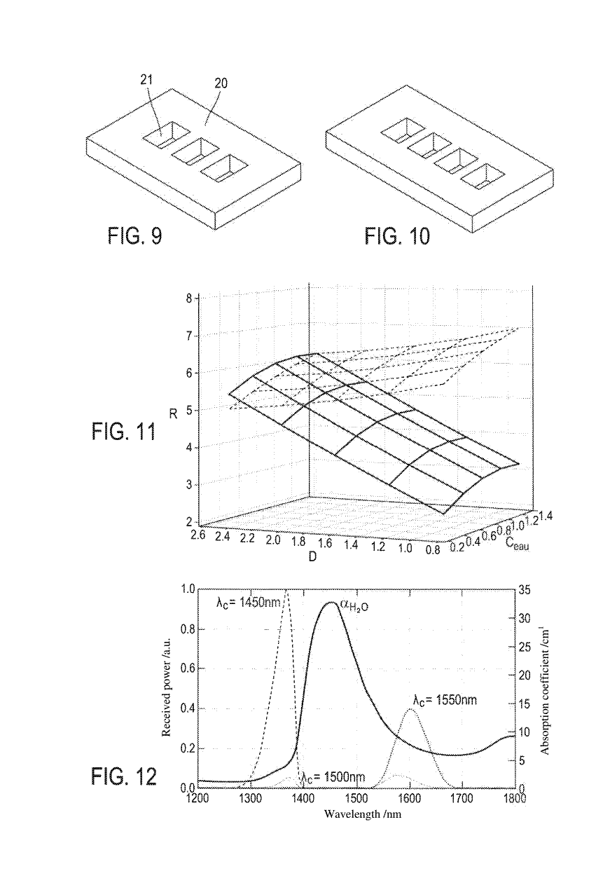

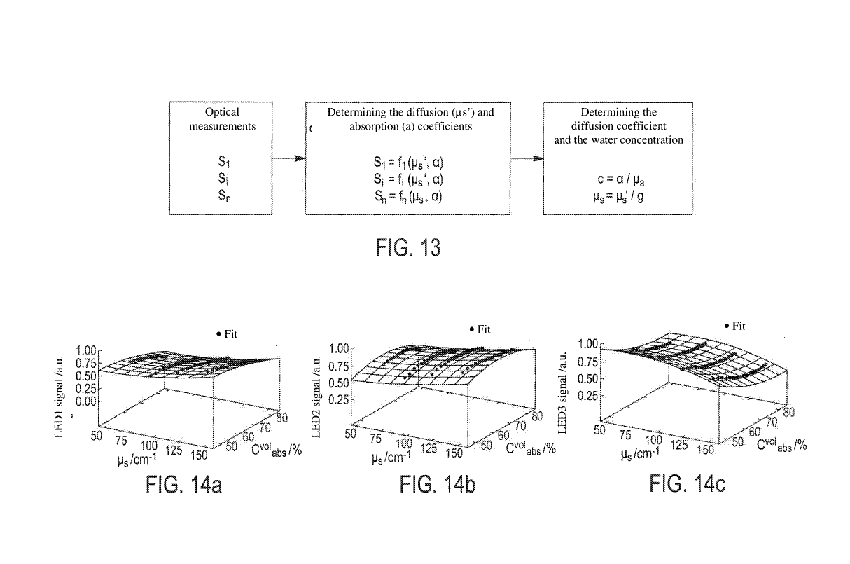

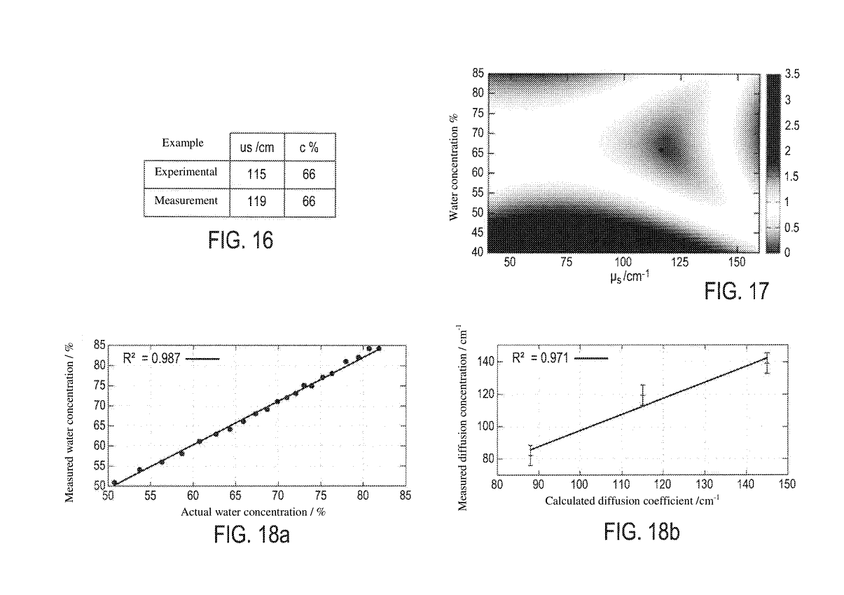

The following describes, in reference to FIGS. 9 to 18, preferred embodiments of the invention. The method according to the invention aims to take a measurement of the optical-diffusion coefficient and the water concentration of a skin sample from light signals collected following an interaction between an incident light and a relatively shallow zone of the skin (a depth less than a maximum depth of less than 2 millimetres, preferably less than 500 microns), signals which are converted into an “absorption coefficient/diffusion coefficient” pair by means of functions pre-established from reference samples of reconstituted skin (referred to as phantom samples) with optimised constitution or from digital models that solve the physical radiative transfer equations that govern the interaction between the sample and the light emitted by the invention and calculate the received power. By being limited to shallow skin depth, there is limited interaction between the light passing through the shallow zone of the skin and elements (venous network, muscles or bones) that are at least as disrupting as the components of the skin, if not more so, to the progression of the incident light, and thus interfere with the measurement of water concentration.

In the case that the maximum depth is around 500 microns, the probed zone is then mainly the dermis and the measurements taken can then be associated with the collagen in the dermis and the water concentration of the dermis. In the case that the maximum depth is around 100 microns, the probed zone is then mainly the epidermis and the measurements taken can then be associated with the keratin in the epidermis and the water concentration of the dermis. A particular choice of types and number of sources of incident light having specific wavelengths and detectors of the light collected after interacting with the studied zone of the skin sample makes it possible to limit to light intensity signals the data necessary for determining the absorption coefficient and the diffusion coefficient, while the methods of the prior art use complete spectra, and to analytic functions (defining three-dimensional surfaces of isocontours) the data resulting from the phantom samples or digital simulations, when the methods of the prior art require arrays or libraries taking up several megabytes, gigabytes or terabytes to index the data obtained from their phantom samples.

Specifically, the operating principle of the method according to the invention is as follows:

A first signal S₁ is detected by lighting the skin with a first light source emitting at a known wavelength, such as a water-absorption wavelength (LED, for example) and by collecting the light obtained from the skin by a first detector (photodiode, for example). This first signal is equivalent to the tip of a water-absorption peak at the chosen absorption wavelength, on an absorption spectrum of the sample of the sample acquired as a function of the wavelengths. A second signal S₂, which is equivalent to the bottom of the aforementioned water-absorption peak, is detected by lighting the skin with a second light source emitting at a known wavelength, such as a zero-water-absorption wavelength (LED, for example) and by collecting the light obtained from the skin by the same detector (photodiode, for example). This light source emitting at a zero-water-absorption wavelength defines, with the detector, a first pair for analysing the diffusion of the sample defining a first separation distance between detector and source.

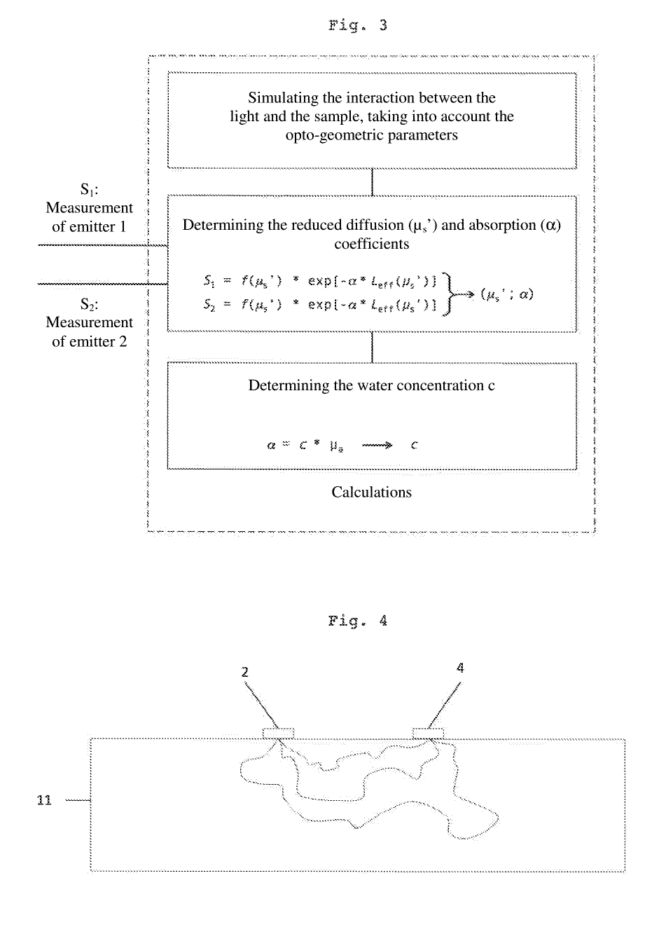

These two first simple measurement signals, which have relatively small storage size, make it possible to obtain information that is equivalent to the height of a water-absorption peak on a spectrum. They can be used to obtain the water-absorption coefficient and the light-diffusion coefficient, according to FIGS. 4 and 5 and the preceding description. However, it is preferable to acquire at least one third signal, since the amount of water in the skin will modify the optical step index with the diffusers and thus modify the light-diffusion coefficient.

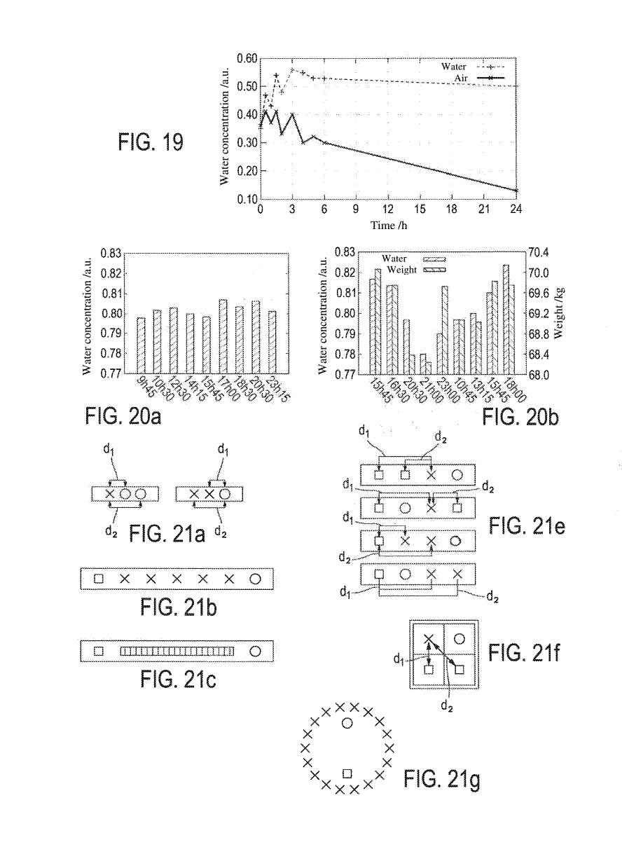

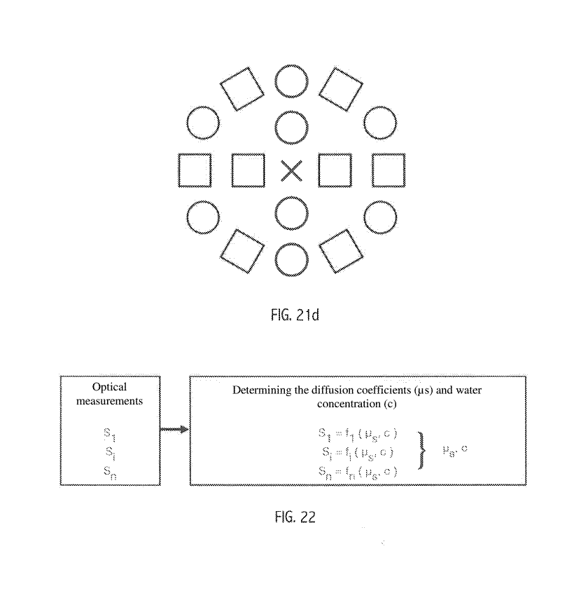

Preferably, therefore, a third signal S3 is detected from a second “light source emitting at a zero-water-absorption wavelength/detector” pair defining a second separation distance, different from the first. The source/sensor pairs can be defined in several ways from various arrangements of sensors/light sources of apparatuses according to various configurations of the invention, certain examples of which are shown in FIGS. 21a to 21g and will be described in detail below.

Execution of Theoretical Models

By resolving the radiative transfer equation for an arrangement of sources and sensors by means of Monte Carlo algorithms, for example, for simulations in which the water concentration and the diffusion coefficient vary, the three signals received (S₁: signal resulting from lighting the phantom sample by a source emitting at a water-absorption wavelength, S₂: signal resulting from lighting the phantom sample by a source emitting at a zero-water-absorption wavelength defining a first distance with the sensor, S₃: signal resulting from lighting the phantom sample by a source emitting at a zero-water-absorption wavelength defining a second distance with the sensor) are calculated. Equations for each signal can then be found in order to obtain the correct compliance between these calculations and the conditions of water concentration and optical-diffusion coefficient. These equations can refer, as non-restrictive examples, to polynomial, exponential, logarithmic or hyperbolic functions.

Chart Production

From samples having known water concentration and known diffusion coefficient, referred to as “phantom samples”, for a given arrangement of sources and sensors of an apparatus according to the invention, charts are produced for the three measured signals (S₁: signal resulting from lighting the phantom sample by a source emitting at a water-absorption wavelength, S₂: signal resulting from lighting the phantom sample by a source emitting at a zero-water-absorption wavelength defining a first distance with the sensor, S₃: signal resulting from lighting the phantom sample by a source emitting at a zero-water-absorption wavelength defining a second distance with the sensor). Charts are shown in FIGS. 14a to 14c, as obtained for the first example of sensor/source arrangement of FIG. 21e.

A three-dimensional representation of the obtained measurements shows that for each of the three signals S₁, S₂, S₃ it is possible on the bases of the trios obtained for the various phantom samples (signal S₁, S₂ or S₃ measured, absorption coefficient, diffusion coefficient) to define relatively simple functions, and in this regard the signals S₁, S₂ and S₃ obtained for all the phantom samples can each be shown by a continuous surface having a progression or curve that can be interpreted scientifically:

progression S₁: for phantom samples having a low absorption coefficient and a low diffusion coefficient, the signal S₁ is relatively constant, since the absorption and the diffusion compensate for one another, and then the signal S₁ falls for higher absorption coefficients, with absorption prevailing over diffusion,

progression S₂: for low water concentrations, the diffused light signal rises with the diffusion coefficient of the phantom samples, and then falls when the absorption coefficient of the samples rises, since the concentration of the diffusers then falls

progression S₃: with a constant absorption coefficient, the signal falls when the diffusion coefficient rises, the incident light being more diffused in the body of the sample as the number of diffusers rises; with a constant diffusion coefficient, the signal falls as the water concentration rises.

This result is remarkable since it makes it possible to define simple analytic functions for each signal S₁ which are obtained by direct measurements on the phantom samples. And for a skin sample having unknown absorption and diffusion coefficients, it suffices to measure, using the same apparatus as that which made it possible to define these charts, the three signals S₁, S₂, S₃, to position these values on the charts and, if there is a unique (absorption coefficient, diffusion coefficient) pair simultaneously fulfilling the three analytic functions defined by the charts, it is possible to equate this unique pair with to the water concentration and the diffusion coefficient of the unknown sample, for example using a least square minimisation technique.

FIG. 18a shows the correct correlation obtained for test samples having fixed diffusion coefficients, between the measured water concentration and the known water concentration thereof. FIG. 18b shows the correct correlation obtained for test samples having a given water concentration, between the measured diffusion coefficient and the known diffusion coefficient thereof. The prepared charts can thus be used to determine the water concentration and the diffusion coefficient of unknown skin samples, while requiring a minimum memory space for the storage thereof, compared with the data arrays or libraries of the known methods.

Device with Three Signals

This type of device makes it possible to obtain an estimate of the water concentration of an unknown sample and that of the light-diffusion coefficient thereof from which it is possible to obtain information on the quality/amount of the collagen or the keratin thereof.

### General Configuration

According to FIGS. 21e and 21f, a device of this type is provided at least with:

a first pair of components formed by a light source (LED) emitting a light spectrum close to a water-absorption peak having a raised effective absorption coefficient, and a sensor (photodiode), defining a first separation distance between the sensor and the source;

a second pair of components formed by a light source (LED) emitting a light spectrum centred around a low- or zero-water-absorption wavelength (e.g. 1300 nm, see spectrum of the water in FIG. 12), and a sensor (photodiode), defining a first separation distance between the sensor and the source;

a third pair of components formed by a second light source (LED) emitting a light spectrum centred around a low- or zero-water-absorption wavelength (e.g. 1300 nm see spectrum of the water of FIG. 12), and a sensor (photodiode), defining a second separation distance.

The choice of wavelengths of the light sources, which are generally LEDs, is guided by the following considerations:

The water concentration in the skin is around 65%. Water has a spectrum shown in FIG. 12, having an absorption peak centred around 1450 nm and having medium absorption. This is the absorption peak that will preferably be used for the needs of the invention.

To this end, knowing that LEDs have a not-inconsiderable spectral width in this spectral range, when choosing the LED as first or second light source making it possible to determine the water concentration, it is advisable to choose an LED having an emission peak centred on a wavelength higher than the chosen water peak (1450 nm). This makes it possible to avoid an excessive overflow towards 1350 nm obtained with a source having an emission peak centred on a wavelength lower than 1450 nm (curve identified by ë_{c}=1450 nm in FIG. 12). Sources having emission peaks centred on 1500 nm or 1550 nm thus make it possible to improve the dynamics and will be preferred. For the third source making it possible to characterise the diffusion, an emission wavelength outside of the water-absorption peak, which is considerably lower than the latter, for example 1300 nm, is chosen.

### Different Possible Arrangements

Aforementioned FIGS. 21e and 21f show different arrangements that comply with these requirements:

The first two devices of FIG. 21e and the device of 21f made up of a sensor (represented by a cross), two sources centred around a zero-water-absorption wavelength (each shown by a square) and defining two respective measurement distances (d1, d2) with the sensor, and a source having a spectrum close to a water-absorption peak, positioned as close as possible to the sensor, preferably at the distance d1 (and represented by a circle). The last two devices of FIG. 21e made up of two sensors (represented by a cross), two sources centred respectively around a zero-water-absorption wavelength (shown by a square), and a source having a spectrum close to a water-absorption peak, positioned as close as possible to the sensor. The two sensors define, with the source centred around a zero-water-absorption wavelength, two measurement distances (d1, d2).

The improved arrangements of FIG. 8, 21b, 21c or 21d make it possible to obtain a plurality of signals representing the water absorption that takes place on different layers of different depths of the studied sample by means of multiple pairs defined between the source emitting around a water-absorption wavelength and the various sensors, as well as a plurality of signals representing the diffusion of the particles on different layers of different depths of the sample studied by means of the multiple pairs defined between the source emitting around a zero-water-absorption wavelength and the various sensors. The linear strip of photodiodes of FIG. 21c makes it possible to obtain practically continuous information on the water concentration and the quality of the collagen and/or the keratin over the entire depth of the zone studied. The arrangements proposed by the devices of FIG. 21g, wherein the sources or the sensors (photodiodes) are distributed in a ring, around an off-centre sensor or an off-centre source (or two separate sources in accordance with the illustration), respectively, so as to define different analysis distances d1 and confirmation measurements. It is possible to provide, for further information on a zone, a multitude of concentric rings as shown in FIGS. 8 and 21d.

### Use of the Information Relating to the Diffusion Coefficient

The information obtained after implementing the method according to the invention on the diffusion coefficient can be used to obtain qualitative information on the collagen (organised into relatively parallel long fibres or disorganised in anarchic vermicelli) or on the keratin as a function of the measurement depth. The various arrangements of devices allowing measurements of diffusion coefficients of a sample in a plurality of directions (FIG. 8, 21d, 21g) can be used to obtain information on the qualitative and quantitative progression of the sample in the various directions of the sample studied. The display means may present information relative to the collagen or the keratin in addition to information relative to the water concentration.

### Mask

The device advantageously comprises a mask as shown in FIGS. 9 and 10 for the arrangements with three elements of FIG. 21a and for the arrangements with four elements of FIG. 21b. This mask is intended for being physically inserted between a source and an adjacent sensor to prevent the direct lighting thereof, and also forms, around each source, a tunnel for concentrating the emitted light in a specific zone.

This mask 20, is formed by a solid block, made of material that is opaque to the wavelengths used, which comprises through-windows 21 in the thickness of the block, open on the large faces thereof. It can be made of plastic or metal material. The windows and/or the beams defined therebetween, having a width of 50 microns to 1 mm, can be obtained by precision mechanics via plastic injection, laser cutting or spark-erosion cutting. This mask is applied to the device for which it was designed, so as to align the windows thereof with the sources and detectors on the device, and then a resin made of biocompatible material is applied to the mask to fill in the windows, and then cured and polished to obtain the desired dimensions and finish. This resin may be coated with a layer of silicone- or oil-based gel protected by a removable film to be removed in order to take a measurement on the skin of an individual.

### Preparation of Optimised Phantom Samples

The possibility of defining relatively simple analytic functions having particularly small storage sizes, from said charts, can be linked with the choice of the number of signals measured, with the low depth of the analysed skin sample, and also with the actual nature of the prepared phantom samples from which the charts, and indirectly the measurements, are produced. The following description explains the issues encountered in the known phantom samples and the solution proposed by the invention. The lack of knowledge on the components of a biological material to be studied, and the concentrations of same therein very often lead to the production of reference samples (or phantom samples) for producing proofs of concept or calibrating devices. These phantom samples, produced artificially, have the aim of being structurally similar to the actual material to be studied, but comprising components of known nature and concentration allowing the qualification or calibration of novel devices or novel measurement methods.

In the case of characterising biological material by the concentration of light diffusers and absorbers therein, the following parameters need to be taken into account:

the optical refractive index of the diffusers,

the shape of the diffusers or the shape distribution thereof,

the size of the diffusers or the size distribution thereof,

the specific weight of the diffusers,

the volume concentration of the diffusers,

the optical refractive index of the environment that surrounds the diffusers (the solvent),

the specific weight of the solvent,

the volume concentration of the one or more light absorbers that are diluted in the solvent.

Specifically, when studying the water concentration and the optical diffusion of the dermis, produced mainly by the collagen, phantom samples made up of the following are usually produced:

diffusing particles in powder form (titanium dioxide or silicon dioxide),

a solvent formed by a water/diluent mixture that is miscible with water and which has very low absorption at the working wavelengths, for example such as dimethyl sulphoxide or deuterium.

The phantom samples produced to simulate the skin often overlook the influence of the anisotropy of the diffusion by choosing diffusing particles that poorly reproduce the value thereof (around 0.9 in the case of the skin). However, these known samples do not satisfactorily reproduce the anisotropy of the diffusion due to the chosen diffusing particles, which poorly reproduce the value thereof (around 0.9 in the case of the skin). They also do not correctly reproduce the skin in terms of optical diffusion since this phenomenon lies in the actual skin, on the variation of the optical index between the diffusing particles and the water, and in the phantom sample, not only like for the skin on the optical index variation between the particles and the added water, but also and in a parasitic fashion, on the optical index variation between the particles and the diluent solvent used to simulate the biological environment of the skin.

Thus, the measurements taken do not reproduce real conditions and induce, at best, an deviation with respect to the in vivo measurements and, at worst, a complete lack of compliance between the measurements taken on the phantom samples and the measurements taken under real conditions. Any calibration of devices is thus rendered obsolete. The aim of the invention was to overcome these drawbacks in order to obtain phantom samples that reproduce, as reliably as possible, in particular in terms of anisotropy and index variation with the absorbent, the optical behaviour of the material to be analysed. Specifically in the case of the skin, this consists of reproducing the optical behaviour of the dermis or the epidermis.

This aim is achieved by:

Using diffusing particles having a shape, size (or their distributions) and step index with the solvent making it possible to reproduce the anisotropy of the environment to be studied.

Adding an additional component to the solvent in order for the index variation between the particles and the solvent to only come from adding the absorbent (water) to the solvent.

The steps of the method for manufacturing test samples of the invention comprise:
a) mixing a diluent and diffusing particles,
b) adding an intermediate component to cancel out the diffusion of particles in the diluent, which will modify the optical index of the diluent in order to make it equal to that of the particles and make the mixture transparent to the working wavelengths (in the non-diffusing direction),
c) adding the absorbent.
   Once the quartet of diluent/particles/absorbent/intermediate component is found, various phantom samples are produced having different known amounts of absorbent and particles.

Determination of the Components of the Reference Sample

a) Choice of absorbent: the absorption spectrum, the refractive index and the specific weight thereof must be as close as possible to those of the absorbent to be studied. It is sometimes possible to use the absorbent in the material to be analysed directly, in this case water or deionised water for the example of studying the water concentration of the skin.

b) Choice of diluent: the refractive index and the specific weight thereof must be as close as possible to those of the absorbent, while the optical absorption thereof in the studied range of wavelengths (water-absorption wavelength and zero-water-absorption wavelength) must be negligible compared with that of the absorbent. It is sometimes possible to use the isotopes of the absorbent.

c) Choice of diffusing particles: the refractive index and the specific weight thereof must be as close as possible to that of the diffusers under real conditions (the collagen or the keratin in the case of studying the skin). Moreover, the size (or the size distribution) thereof must make it possible to obtain similar anisotropy to that of real conditions.

d) Choice of intermediate component: during the addition thereof to the diluent/absorbent mixture, the addition of an intermediate component must make it possible to equalise the refractive index of the diluent and the refractive index of the diffusing particles before adding the absorbent.

It can be noted that steps a), b) and c) are interchangeable.

The aim is to obtain absorbent concentrations, diffusion coefficients and anisotropy that are as similar as possible to actual conditions:

the absorbent concentration is directly controlled by the amount of absorbent added to the solvent,

the diffusion coefficient and the anisotropy are controlled by optical diffusion models that makes it possible to calculate them: Mie diffusion, Rayleigh diffusion, case of spherical particles, etc.; and to determine given particle shapes and sizes for a diffusion coefficient and an anisotropy to be achieved,

in addition to needing to be transparent to the working wavelengths, the choice of intermediate component is mainly guided by the choice of the refractive index thereof, which is given by the following formula:

nₚ=cᵢ*nᵢ+c_{d}*n_{d}

or nᵢ=(nₚ−c_{d}*n_{d})/cᵢ

wherein nₚ, nᵢ and n_{d} are the refractive indices of the diffusing particles, the intermediate component and the diluent, respectively, and cᵢ and c_{d} are the mass concentrations of the intermediate component and the diluent in the mixture, respectively.

### Example: Case of the Dermis

This principle of making the mixture of diluent and particles transparent to the working wavelengths can be transposed to different types of materials to be simulated by phantom samples in order to study different types of components that absorb light. In the case of simulating the dermis by phantom sample, to assess the water content and the state of the collagen of unknown samples, this dermis can, according to the method for preparing phantom samples according to the invention, be modelled by a set of diffusing particles (collagen) which is submerged in an absorbent medium (water).

In order to reproduce the optical features of the dermis, it is possible, for example, to use a mixture that comprises:

water (absorbent),

heavy water (diluent)

spherical particles of titanium dioxide or silicon with a diameter centred around 1.5 pm.

hydrazine or pyridine (intermediate component)

This combination makes it possible easily to obtain adapted diffusion coefficients (50/cm->150/cm) for conventional water concentrations in the dermis (50%->80%) and with an anisotropy of 0.89 (in the context of Mie diffusion in the presence of spherical particles). Other combinations are also foreseeable, in particular as regards the choice of the intermediate component.

Formation of Solid Phantom Samples

An advantageous embodiment consists of making the mixture solid. For this purpose, steps of solidification with hardening agents (such as silicone, for example), elastomers (such as silicone, for example) or gelling products (such as gelatine or agar agar, for example), optionally with annealing and degassing steps, are added.

Proofs of Concept

Ex vivo measurements (FIG. 19) have been taken on samples of pig skin left to dry in the open air and skin in contact with physiological liquid using the device of FIG. 21e). The water concentrations of the samples left exposed to the air, as supplied by the method according to the invention, fall as a function of the air exposure time, showing the gradual drying thereof, while those of the sample in contact with the physiological liquid are relatively constant over time, showing the consistency of their water content. In vivo measurements taken using the device of FIG. 21e on a volunteer patient (dermis of the calf), during a day with no particular sporting activity (FIG. 20a) as well as during a day interrupted by a sports session at a specific time (4:30 PM), after which the volunteer did not drink anything until 9:30 PM and then had dinner, by the method according to the invention show the consistency of the water concentration measured (FIG. 20a), and the fall of the water concentration following the sports session followed by a gradual recovery (FIG. 20b), said variation being confirmed by the measurement of the weight of the volunteer, which indicates a water loss of around 3% and a weight recovery after dinner.

The invention claimed is:

1. A method for measuring water concentration in a light-diffusing material comprising:
emission by N light sources of beams having wavelengths onto the material;
acquisition by M sensors sensitive in at least one portion of the wavelengths of signals emitted by the material;
wherein M+N≥3;
calculating a first piece of information representing diffusion, and a second piece of information representing absorption, as a function of the signals and a piece of digital information representing the diffusing material and the sources as well as the sensors, the piece of digital information being provided by one or more functions which are determined by calibration by signal acquisition for samples having a known water content; and
calculating the water concentration in the sample as a function of the second piece of information.

2. The method for measuring the water concentration according to claim 1, wherein the wavelength is chosen to be close to 1450, 1940, 970 or 1190 nanometres, for the light sources and sensors outputting a signal representing the diffusion in a wavelength corresponding to a water-absorption peak.

3. The method for measuring the water concentration according to claim 1, wherein the wavelength is chosen to be close to 1050, 1070, 1100 or 1300 nanometres, for the light sources and sensors outputting the signal representing the diffusion in a wavelength other than a water-absorption peak.

4. The method for measuring the water concentration according to claim 1, wherein the piece of digital information representing the diffusing material is made up of a digital model defining analytic functions obtained from optimised phantom samples.

5. The method for measuring the water concentration according to claim 1, wherein the piece of digital information representing the sources comprises information chosen among geometry of the sources and optical characteristics of the sources, and the piece of digital information representing the sensors comprises information chosen among the geometry of the sensors and the optical characteristics of the sensors.

6. The method for measuring the water concentration according to claim 1, wherein:
the piece of digital information is provided by one or more functions which are determined by determining diffusion and absorption coefficients from a digital model for simulating the light/sample interaction; and
the piece of digital information representing the diffusing material is made up of a digital model defining analytic functions obtained from digital simulations.

7. An apparatus for measuring the water concentration in a light-diffusing material comprising:
N light sources emitting in wavelengths;
M sensors sensitive in at least one portion of the wavelengths, wherein N+M≥3; and
an electronic circuit including, for each of the sensors, a signal-processor outputting a signal and a computer controlling an output process, as a function of the signals and a piece of digital information representing diffusing material and geometry of the sources and of the sensors, the piece of digital information being supplied by one or more functions which are determined by calibration including acquiring signals for samples having a known water content, a first piece of information representing the diffusion, and a second piece of information representing the absorption, and processing to determine the water concentration.

8. The apparatus according to claim 7, wherein the piece of digital information is supplied by one or more functions which are determined by determining diffusion and absorption coefficients from a digital model for simulating the light/sample interaction.

9. The apparatus according to claim 8, wherein the light sources and the sensors are inserted into a mounting opening by a window for contact with the material to be characterised, the mounting comprising a screen that prevents a direct and/or reflected transmission, without prior penetration into the diffusing material, of light between the light source and the sensor, and the window of the mounting has an anti-glare treatment.

10. The apparatus according to claim 7, wherein the light sources are polarised and a direction of the emitted beam forms a Brewster angle with an optical axis of the sensors.

11. The apparatus according to claim 7, further comprising a radiocommunicator periodically transmitting a result of a measurement to a remote apparatus.

12. The apparatus according to claim 7, further comprising a patch having a reversible connection with a patient.

13. The apparatus according to claim 7, further comprising at least two light source/sensitive sensor pairs, a first light source/sensitive sensor pair defining a first separation distance between the sensor and the source, and a second light source/sensitive sensor pair defining a second separation distance.

14. The apparatus according to claim 13, wherein:
the sources of the two pairs both emit with a spectrum centred around a wavelength close to or higher than a water-absorption wavelength or in that one of the two sources emits with a spectrum centred around a wavelength close to a water-absorption peak and the other source emits with a spectrum centred around a zero-water-absorption wavelength; and
the sources of the two pairs both emit with a spectrum centred around an absorption wavelength of 1450 nm, 1500 nm or 1550 nm.

15. (canceled)

16. The apparatus according to claim 13, wherein:
the sources of the two pairs both emit in a zero-water-absorption wavelength; and
the sources of the two pairs both emit with a spectrum centred around 1300 nm.

17. (canceled)

18. The apparatus according to claim 16, further comprising:
a third light source/sensor pair, the light source emitting in a water-absorption wavelength;
the light source emitting in a water-absorption wavelength is arranged as close as possible to the sensor; and
a sensor and three sources, the three sources belonging to three separate source/sensor pairs, respectively, or comprising two sensors and two sources, one of the two sources emitting in a wavelength close to a water-absorption peak, and the other source emitting in a zero-water-absorption wavelength.

19-20. (canceled)

21. The apparatus according to claim 14, further comprising, between the two separate sources, a series of separate sensors arranged in line.

22. The apparatus according to claim 14, further comprising, between the two sensors, a linear strip of photodiodes.

23. The apparatus according to claim 13, further comprising a central or off-centre sensor and, around this sensor, a series of light-emitting sources centred around absorption and/or zero-absorption wavelengths of light.

24. The apparatus according to claim 13, further comprising at least one central or off-centre light-emitting source emitting with a light spectrum centred around a absorption or zero-absorption wavelength of light and, around this source, a series of sensors.

25. The apparatus according to claim 7, further comprising a mask inserted physically between a source and an adjacent sensor, the mask creating around each source a tunnel operably concentrating emitted light in a separate zone.

26. (canceled)

27. The method according to claim 1, further comprising characterizing the water concentration in at least one of: a plant, a cellulosic matrix like paper, cardboard, wood, a construction material, a building coating, a fabric or leather.

28. The method according to claim 1, further comprising characterizing at least one of: hydration of skin; hydration of a lock of hair; or a quality and/or the amount of collagen or keratin in the skin.

29. The method of claim 1, further comprising characterizing the water concentration in at least one of: a hydrocarbon; a chemical product; a powder; in a food matrix; or in a medicinal product.
